(19) United States
(12) Patent Application Publication
(10) Pub. No.: US 2009/0326385 A1
(43) Pub. Date: Dec. 31, 2009

(54) OBTAINING OPTICAL TISSUE PROPERTIES

(75) Inventors: Bernardus Hendrikus Wilhelmus Hendriks, Eindhoven (NL); Stein Kuiper, Eindhoven (NL); Ruth Wilhelmine Ireen De Boer, Eindhoven (NL); Augustinus Laurentius Braun, Eindhoven (NL); Michael Cornelis Van Beek, Eindhoven (NL); Wouter Harry Jacinth Rensen, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/517,249

(22) PCT Filed: Nov. 29, 2007
(86) PCT No.: PCT/IB07/54846

§ 371 Date: Jun. 2, 2009

(30) Foreign Application Priority Data
Dec. 6, 2006 (EP) 06125518.8

(51) Int. Cl. A61B 6/00

(52) U.S. Cl. 600/478

(57) ABSTRACT

This application describes a medical device (230) for obtaining optical tissue properties of a target material. The medical device (230) comprises an elongated body (231) having a longitudinal axis (232) and an optical fiber being integrated within the elongated body (231). The optical fiber has a second fiber end (242, 242a, 242b), which is arranged at a side wall (233) of the elongated body (231) and which provides a lateral field of view with respect to the longitudinal axis (232). According to an embodiment many optical fibers are integrated each having an optical outlet (242, 242a, 242b) around the elongated body (231). Using the outlets (242, 242a, 242b) to do diffuse optical tomography and also use optical fibers to do an optical inspection, one can get information on the presence of tumors in a volume around the medical device (230) and a tissue characterization in the vicinity of the medical device (230). Thereby, an optical biopsy may be carried out, wherein no real tissue is removed. According to another embodiment an optical detection system is integrated into a real biopsy needle (330) allowing inspection and taking real biopsy simultaneously.

### FIELD OF INVENTION

The present invention relates to the field of medical tissue examinations. In particular, the present invention relates to a medical device such as a needle for obtaining optical tissue properties of a human or animal body. The medical device, which is insertable into tissue to be probed, comprises at least one optical fiber for directing illumination light to the tissue and for receiving measurement light having interacted with the tissue. The present invention further relates to a medical apparatus and to a method for obtaining optical tissue properties of a human or animal body. Both the medical apparatus and the method take benefit from the described medical device.

### ART BACKGROUND

For correct diagnosis of various cancer diseases biopsies are taken. This can either be via a lumen of an endoscope or via needle biopsies. Biopsies may be taken for instance from the prostate via the rectum. In order to find the correct position to take the biopsy, various imaging modalities are used such as X-ray, magnetic resonance imaging and ultrasound. In case of prostate cancer in most cases the biopsy is guided by ultrasound. Although helpful, these methods of guidance are far from optimal. The spatial resolution is limited and, furthermore, these imaging modalities can in most cases not discriminate between benign and malignant tissue. As a result, during a biopsy procedure one does not know whether a specimen is taken from the correct part of the tissue. This means that typically more or less blind biopsies are carried. This has the effect that even if after inspection of the tissue no cancer cells are detected, one does not know for sure that one did not simply miss the right spot to take the biopsy. Therefore, in order to improve the accuracy, the number of needle biopsies taken can be increased. However, since each biopsy causes a scarf and possibly complications, this is not a preferred solution.

US 2005/0203419 A1 discloses a needle biopsy, which includes the step of inserting an optical spectroscopy probe in the needle and gathering optical information through a window formed in the side of the needle at its distal end. The optical probe includes an illumination optical fiber, which conveys light to the tissues adjacent the side window and a detection optical fiber, which collects light from the same tissues and conveys it to an optical spectroscopy instrument. Based on the results of the optical spectroscopy measurement, the optical probe may be withdrawn from the needle and a cutter advanced to acquire a sample of the tissues adjacent the side window.

U.S. Pat. No. 5,318,023 discloses a method and an apparatus for the instant intra-operative detection and biopsy of metastatic cancer using fluorescence spectroscopy. A photosensitizing agent selectively retained by cancerous tissue is administered prior to surgery. A fiber optic probe integrated with a biopsy device illuminates the examined tissue and causes fluorescence, which is recorded by a spectrograph and plotted as a spectral curve.

US 2005/0027199 A1 discloses a method and an apparatus for identifying tissue structures in advance of a mechanical medical instrument during a medical procedure. A mechanical tissue penetrating medical instrument has a distal end for penetrating tissue in a penetrating direction. An optical wavefront analysis system provides light to illuminate tissue ahead of the medical instrument and receives light returned by tissue ahead of the medical instrument. An optical fiber is coupled at a proximal end to the wavefront analysis system and attached at a distal end to the medical instrument proximate the distal end of the medical instrument.

There may be a need for providing a tool and a method for obtaining more detailed optical tissue properties of a human or animal body.

### SUMMARY OF THE INVENTION

This need may be met by the subject matter according to the independent claims. Advantageous embodiments of the present invention are described by the dependent claims.

According to a first aspect of the invention there is provided a medical device, in particular a needle, for obtaining optical tissue properties of a human or animal body. The medical device comprises (a) an elongated body having a longitudinal axis, wherein the elongated body is designed to be insertable into tissue of a human or an animal body, and (b) an optical fiber being integrated within the elongated body. The optical fiber has a first fiber end and a second fiber end, wherein the first fiber end is adapted to be coupled to an optical instrument, wherein the second fiber end is arranged at a side wall of the elongated body and wherein the second fiber end provides a lateral field of view, which is directed in a lateral direction with respect to the longitudinal axis.

This aspect of the invention is based on the idea that by providing a lateral field of view, which is directed in a sidewise direction with respect to the elongated body, the effective lumen, which can be investigated with the described medical device, may be significantly increased. By contrast to known medical devices being equipped with optical fibers, which are directed in a longitudinal direction of an elongated body only and which comprise an optical fiber end at a frontal respectively distal tip, the described medical device may allow for optically investigating tissue being located aside from the branch canal. Thereby, the term branch canal is used for the substantially tubular opening, which will at least temporarily develop within the tissue when the medical device is inserted into the tissue.

It has to be mentioned that the term lateral field of view means that the field of view is not directed solely in a direction being oriented parallel with respect to the longitudinal axis. In this respect lateral field of view rather means that (a) the beam path of light originating from the second fiber end of the optical fiber or (b) the beam path of light impinging onto the second fiber end of the optical fiber is oriented angularly with respect to the longitudinal axis of the elongated body. Preferably, these beam paths are oriented at least approximately at a right angle of 90° with respect to the longitudinal axis. However, also other angles deviating from 0° and 90° may be possible in order to optically investigate tissue being located sidewise with respect to the elongated body.

Further, it has to be mentioned that optical fiber may be adapted (a) to transmit illumination light from the optical instrument to the tissue, (b) to transmit measurement light from the tissue to the optical instrument or (c) to transmit both illumination light in a first direction and to transmit measurement light in a second direction being opposite to the first direction. In the latter case appropriate beam splitting means have to be provided for instance at a proximal fiber end of the medical device or at the corresponding optical instrument in order to allow for a spatial separation of the measurement light from illumination light.

By sequentially moving the medical device through the tissue of a patient's body one can obtain information regarding optical properties of a large tissue lumen being located sidewise from the medical device. Thereby, also a rotational movement of the medical device might be carried out in order to optically investigate tissue in different angular directions with respect to the longitudinal axis.

It has to be pointed out that the described medical device is not restricted to be inserted into tissue of a patient under examination. The described medical device can also be inserted for instance in a vessel or in other tubular structures of the patient. In general, the described medical device may be inserted into any target material, which is supposed to be optically investigated.

According to an embodiment of the invention the medical device further comprises a reflector element, which is arranged at the side wall of the elongated body and which is optically coupled to the second fiber end of the optical fiber. The described reflector element may provide the advantage that in particular if a thin elongated body is used and/or an angle between the lateral field of view and the longitudinal axis of at least approximately 90° is desired, a strong bending of the optical fiber can be avoided. This makes the manufacturing of the medical device much more easy because the risk of breaking the optical fiber with bending the same is significantly reduced.

The reflector element may be formed integrally with the elongated body. This may provide the advantage that the production of the medical device will be simplified. Alternatively, the reflector element may be formed as a separate optical component, which has to be attached to the elongated body. A separate reflector element may provide the advantage that a very high optical quality of the reflector element can be realized by means of an individual treatment of the reflector element. The reflector element may be for instance a mirror or a prism having a polished surface.

According to a further embodiment of the invention the elongated body comprises a sharpened distal end. This may provide the advantage that the medical device can be inserted into the tissue without significantly infringing respectively hurting the tissue.

According to a further embodiment of the invention the medical device further comprises an optical waveguide being integrated within the elongated body, the optical waveguide having a first waveguide end and a second waveguide end. Thereby, (a) the first waveguide end is adapted to be coupled to an optical instrument, (b) the second waveguide end is arranged at a front end of the elongated body and (c) the second waveguide end provides a front field of view, which is directed in a longitudinal direction with respect to the longitudinal axis. This may provide the advantage that the tissue lumen, which can be optically investigated, can be further increased by also probing the tissue being located directly in front of the elongated body.

Further, when guiding the medical device through a patient's tissue an operating person can optically characterize the tissue into which the medical device is going to be inserted. Thereby, a better navigation of the medical device might be achieved in particular when inserting the medical device in sensitive tissue.

It has to be mentioned that the optical waveguide may also comprise a plurality of optical fibers elements, which represents a whole bundle of individual optical fiber elements. Thereby, the bundle of optical fiber elements may represent an optical imaging system, which allows for obtaining images of the tissue being located in front of the medical device. Thereby, at least some of the individual optical fiber elements may be used for guiding an illumination light into the patient's tissue.

According to a further embodiment of the invention the medical device further comprises at least one further optical fiber being integrated within the elongated body, wherein the further optical fiber has a further first fiber end and a further second fiber end. The further first fiber end is adapted to be coupled to an optical instrument, the further second fiber end is arranged at a side wall of the elongated body and the further second fiber end provides a further lateral field of view, which is directed in a lateral direction with respect to the longitudinal axis.

This may provide the advantage that the tissue lumen being located laterally from the medical device can be simultaneously investigated by means of different optical fibers each having a lateral field of view. Of course, each or at least some of the further second fiber ends may be optically coupled to a respective reflector element in order to eliminate the need for a strong bending of the corresponding fiber optic at the distal end of each further optical fiber.

Preferably, a plurality of second fiber ends respectively further second fiber ends are provided at the side wall of the elongated body. Thereby, the fiber ends may be distributed within a predominately cylindrical shell respectively predominately cylindrical superficies surface of the elongated body. However, the fiber ends may also be distributed on a tapered surface.

If the medical device comprises a plurality of different second fiber ends there are various different possibilities in order to employ these second fiber ends for optically investigating the lateral tissue surrounding the medical device. In the following there will be described as an example three of these possibilities for operating the described medical device:

According to a first possibility one or more of the second fiber ends are used for illuminating the tissue laterally surrounding the elongated body. The illumination light will be scattered by the tissue and at least some photons of the illumination light will be received by at least some of the other second fiber ends. These received photons represent the measurement light, which can be collectively analyzed by means of a spectrometer. In this case the spectral distribution of the measurement light might reveal physiological properties of the overall tissue laterally surrounding the medical device.

According to a second possibility at least some of the second fiber ends or preferably all of the second fiber ends are used both for transmitting illumination light to the tissue and for receiving measurement light, which has been scattered back by the investigated tissue. Thereby, each employed second fiber end has to be coupled both to a common light source for generating the illumination light and to a common light detector for receiving the measurement light. Thereby, a beam splitter might be used for spatially separating the illumination light from the measurement light.

According to a third possibility one of the optical fibers is used for transmitting illumination light such that the corresponding second fiber end represent an illumination source. The illumination light will be scattered by the surrounding tissue and at least some photons of the illumination light will be received by at least some of the other second fiber ends. The received photons again represent the measurement light. However, by contrast to the first possibility, the measurement light is analyzed individually for each optical fiber. Thereby, the analysis might comprise the intensity and/or the spectral distribution of the individually collected measurement light.

In order to acquire even more detailed information regarding the tissue laterally surrounding the medical device the optical fiber, which is used for illumination and as a consequence the spatial position of the activated second fiber end representing the illumination source, can be changed for instance in a sequential manner. Thereby, the measurement is carried out sequentially within different time slots, wherein within each time slot a different second fiber end is activated.

It has to be mentioned that this embodiment allows a three-dimensional (3D) imaging of scattering and absorption properties of the tissue laterally surrounding the medical device. Thereby, a longitudinal resolution equal to that of the distance between neighboring second fiber ends can be achieved.

At this point it has to be mentioned that the described optical scan corresponds to a method, which is called diffusive optical tomography (DOT). DOT is an emerging medical imaging modality. It is a technique in which tissue is illuminated preferably with near-infrared light. The light emerging from the tissue is detected, and by making use of a model of the light propagation in the tissue, the localized optical properties of the tissue are determined. In order to obtain a 3D image, the above tomographic type of measurement may be performed. The tissue to be imaged is illuminated from different source positions, and the light emerging from the tissue is detected from all possible directions. The calculation of the 3D image from these source-detector measurements is called image reconstruction.

DOT allows a functional imaging in a relatively large volume around the medical device similar to what is done in optical mammography, although the imaged volume will be smaller compared to optical mammography due to the measurement configuration in the embodiment described here. In the embodiment described here one or more optical fibers are used for a sequential illumination of the tissue. Further, one or more other optical fibers are used to collect the scattered light. Using an image reconstruction algorithm it is possible to obtain a 3D map of the optical properties in a region around the medical device.

The main advantage of DOT is the high penetration depth compared to other optical methods. In the near infrared spectral regime the penetration depth is at its maximum and the optical properties are strongly determined by important physiologic parameters like blood content and oxygen saturation. By combining DOT at different wavelengths it is possible to translate optical parameters into physiological parameters.

In case a medical device comprising the above-described second waveguide with a second waveguide end being provided at the front end of the elongated body is employed, the described DOT can be extended in such a manner that also this second waveguide end is used for transmitting illumination light and/or for receiving scattered measurement light. This may provide the advantage that also the tissue being located in front of the medical device can be spectroscopically analyzed.

According to a fourth possibility, one can also perform an optical coherence tomography (OCT) scan for each optical fiber. This gives for each optical fiber a depth scan along a line. By combining these lines one can reconstruct a three-dimensional (3D) image of the tissue around the elongated body. Again, a longitudinal resolution corresponding to the distance between neighboring second fiber ends can be achieved.

Further, it has to be mentioned that the four above-described possibilities and any other possibilities for operating the described medical device rely on direct absorption and scattering properties of the tissue under investigation. However, it is also possible to map fluorescence signals of tissue by illuminating with the proper wavelength and simultaneously blocking the illumination wavelength at the detector side. The fluorescence can be endogenous or exogenous, i.e. with the aid of contrast agents. The specificity of the fluorescence detection can be improved by methods well known in the art such as fluorescence lifetime imaging. In fluorescence lifetime imaging a pulsed illumination is used and the temporal decay of excited atoms and/or molecules are used in order to discriminate in time the decayed measurement light from the illumination light respectively the excitation light.

Last but not least it is pointed out that the described medical device can be operated with Raman spectroscopy in order to obtain further characteristic properties of the tissue surrounding the needle. Raman spectroscopy allows distinguishing normal tissue from abnormal tissue. Of course Raman spectroscopy can also be carried out by means of the optical waveguide described above, which optical waveguide extends to the front end of the elongated body.

According to a further embodiment of the invention the elongated body is a solid shaft. This may provide the advantage that a plurality respectively a whole bundle of optical fibers can be accommodated respectively can be integrated in the solid shaft. Due to the additional space available for the optical fibers the number of optical probing points may be significantly increased such that a higher resolution imaging and probing is allowed.

It has to be pointed out that the term “solid shaft” does not necessarily mean that the shaft is made from a solid material. In this respect solid shaft means that the shaft is not hollow in that sense that other components like optical fibers, holder elements like spacer disks may be accommodated within the shaft.

It has to be mentioned that in particular the described medical device having a solid shaft can be used as a so-called optical biopsy device. Thereby, there is no real tissue material removed from a patient's body, which tissue material is also called biopt or specimen. The tissue material is rather investigated in vivo within the patient's body.

According to a further embodiment of the invention the elongated body is a hollow shaft. Thereby, the optical fiber and, if necessary, also the optical waveguide is integrated in the shaft wall. This may provide the advantage that the interior of the hollow shaft can be used as a cannula e.g. for introducing a contrast agent and/or a fluorescence material into the tissue, which is supposed to be optically investigated.

Further, the cannula can be used for applying a photosensitive agent such as amino levulic acid (ALA). ALA may provide the advantage that it is not only applicable for cancer diagnostics, it also constitutes a potential tool for photodynamic cancer treatment, which could also been carried out in vivo by employing the described medical device.

According to a further embodiment of the invention the medical device further comprises a biopsy element being movably accommodated within the hollow shaft. This means that a tool, which is adapted to mechanically interact with the tissue, can be combined in an advantageous manner with the described hollow shaft.

The biopsy element may comprise a recess, which is adapted to accommodate the biopt tissue after the biopt respectively the specimen has been removed. The specimen removal can be supported by a cutting interaction between the recess and a front edge of the hollow shaft representing a blade.

If the medical device is equipped with a second waveguide and a second waveguide end being arranged at the front end of the hollow shaft, it is possible to inspect the specimen material at the tip of the shaft prior to removing it through the shaft. This also allows checking whether the biopsy resulted in sufficient tissue for inspection by a pathologist.

It has turned out that with Raman spectroscopy benign and malignant tissue may be distinguished. Therefore, Raman spectroscopy carried out with the second waveguide end being located at the front end can be used for guiding a biopsy procedure. Thereby, the medical device can be directed in a aimed manner towards the malignant tissue. In this respect it has to be pointed out that accuracy of the diagnosis based on Raman data does not need to be perfect, because the real clinical diagnosis is done later by pathology on the removed specimen.

In other words, Raman spectroscopy merely allows for inspecting the tissue locally before taking the actual specimen. Therefore, the number of needle biopsies can be minimized while the accuracy of the biopsy procedure is actually improved.

According to a further embodiment of the invention the second fiber end provides an interior lateral field of view, which is directed from the shaft wall towards the central longitudinal axis of the hollow shaft. This may provide the advantage that a tissue specimen, which has been removed from the patient's body by means of the biopsy element, may be immediately optically analyzed. Thereby, in a first approximation one can inspect whether the specimen is of good quality and whether the specimen contains sufficient tissue prior to removal. If this is not the case, a new biopsy can be carried out right away before removing the medical device from the patient's body. In this respect one has to take into account that the lumen of the biopsy element comprises enough space to allow more than one specimen to be taken from the patient under examination.

Preferably, the medical device comprises two types of optical fibers. A first type of optical fibers, which have a lateral field of view being directed radially outward from the hollow shaft, and a second type of optical fibers, which have the interior lateral field of view described with this embodiment. Further, the medical device might also be equipped with the optical waveguide extending to the front end and allowing to illuminate and to investigate the tissue being located in front of the distal end of the medical device. Upon inspection of the various spectra obtained with material being located laterally aside from the hollow shaft and/or in front of the hollow shaft, one can decide either to take a biopt or to move the hollow shaft further to another position if no anomalies in the spectrum are found.

According to a further aspect of the invention there is provided a medical apparatus for obtaining optical tissue properties of a human or animal body. The provided medical apparatus comprises (a) a medical device according to any one of the embodiments described above and (b) an optical instrument, which is optically coupled to the optical fiber of the medical device.

According to an embodiment of the invention the optical instrument comprises (a) a light source, which is adapted to generate illumination light for being injected into the optical fiber, and (b) an optical detector, which is adapted to receive measurement light being transmitted by the optical fiber.

In this respect it has to be pointed out that the illumination light and the measurement light might be guided with one and the same optical fiber. In this case, as has already been pointed out above, a beam splitter may be used in order to spatially split the illumination beam path from the measurement beam path such that both the light source and the spectrometer device can be optically coupled to the optical fiber.

Instead of a beam splitter also a so-called pigtail optical fiber may be used, which comprises two first fiber ends. In this case one first fiber end is coupled to the light source and the other first fiber end is coupled to the spectrometer device. Alternatively, for guiding the illumination light a first optical fiber may be used and for guiding the measurement light a second optical fiber may be used.

The light source may be a monochromatic light source such as a light emitting diode or a laser light source. The light source may also be a polychromatic light source such as a light bulb. The light source may also be a combination of different monochromatic and/or polychromatic light sources. The spectral distribution of the light source may be adapted to the appropriate spectral range. A spectral range adjustment may also be carried out by means of appropriate filters.

Further, as has already been described above, the light source may be a pulsed light source, which in cased of a synchronized pulsed light detection may provide the possibility to timely discriminate the measurement light from the illumination light. Of course, such a temporal discrimination would require a decaying de-excitation of atoms or molecules, which have been excited by the pulsed illumination light.

According to a further embodiment of the invention the optical instrument is adapted to perform diffused optical tomography and/or the optical instrument is adapted to perform optical coherence tomography.

Diffused optical tomography is in particular advantageous if a medical device comprising a plurality of optical fibers is used. As has already been described above this may allow for illuminating the tissue under examination from different source positions and detecting the light emerging from the tissue in different directions. Thereby, based on a plurality of different source-detector measurements a 3D image may be reconstructed.

Diffused optical tomography (DOT) may allow for a functional imaging in a relatively large volume around the medical device. Preferably DOT is carried out in the near infrared spectral regime. The near infrared spectral range has a spectral bandwidth between 700 nm and 1400 nm and preferably between 700 nm and 800 nm. In this spectral range the tissue penetration depth is at its maximum and the optical properties of human or animal tissue are strongly determined by important physiologic parameters like blood content and oxygen saturation.

According to a further embodiment of the invention the optical instrument is adapted to perform at least one of the following optical procedures: Raman spectroscopy, fluorescence spectroscopy, auto fluorescence spectroscopy, two-photon spectroscopy, and differential path length spectroscopy. This may provide the advantage that the above-described medical device may be used for applying a plurality of different optical procedures.

For instance Raman spectroscopy may provide a measure of the molecular composition of tissue. By using an appropriate algorithm one can distinguish between benign and malign prostate biopsies with an overall accuracy of 89%. For further details reference is made to the publication “The use of Raman spectroscopy to identify and grade prostatic adenocarcinoma in vitro; P. Crow, N. Stone, C. A. Kendall, J. S. Uff, J. A. M. Farmer, H. Barr and M. P. J. Wright; British Journal of Cancer (2003) 89, 106-108”. The disclosure of this publication is hereby incorporated by reference.

For instance differential path length spectroscopy (DPS) may be used to determine the local optical properties of e.g. breast tissue in vivo. DPS measurements may yield information on the local tissue blood content, the local blood oxygenation, the average micro-vessel diameter, the β-carotene concentration and the scatter slope. Thereby, malignant breast tissue can be characterized by a significant decrease in tissue oxygenation and a higher blood content compared to normal breast tissue. For further details reference is made to the publication “Optical biopsy of breast tissue using differential path-length spectroscopy; Robert L P van Veen et al (2005) Phys. Med. Biol. 50 2573-2581”. Also the disclosure of this publication is hereby incorporated by reference.

According to a further aspect of the invention there is provided a method for obtaining optical tissue properties of a human or animal body. The provided method comprises (a) illuminating the tissue with illumination light, which has been emitted from a light source and which has been transmitted by means of a medical device as described above, and (b) detecting measurement light, which has interacted with the tissue and which has been transmitted by means of the medical device.

This aspect of the present invention is based on the idea that by using the above-described medical device a significant enlarged lumen may be optically investigated. This is based on the matter of fact that by contrast to prior art medical devices, which allow only an investigation of tissue being located in front of a distal front end of the medical, the described method allows for investigating tissue material laterally surrounding the medical device.

The measurement light may be analyzed by means of a spectrometer device, which is capable of measuring the spectral distribution of the measurement light.

At this point it has to be emphasized that the described method for obtaining optical tissue properties is not used for providing a diagnosis or about treating patients. The described method and all other aspects and embodiments of the present inventing merely provide additional and more detailed information, which may assist a physician in reaching a diagnosis and/or in deciding about appropriate therapy procedures.

According to an embodiment of the invention the method further comprises applying a photosensitive agent. This may be in particular advantageous in connection with fluorescence spectroscopy. Fluorescence spectroscopy may allow for a clear identification of certain tissue material in particular if the photosensitive agent has an affinity to this tissue material.

Photosensitive agent can be used not only for cancer diagnosis. If the photosensitive agent also comprises photodynamic properties it can also be used for photodynamically treating for instance carcinogenic tissue. The photosensitive agent may be for example amino levulic acid (ALA).

It has to be noted that embodiments of the invention have been described with reference to different subject matters. In particular, some embodiments have been described with reference to apparatus type claims whereas other embodiments have been described with reference to method type claims. However, a person skilled in the art will gather from the above and the following description that, unless other notified, in addition to any combination of features belonging to one type of subject matter also any combination between features relating to different subject matters, in particular between features of the apparatus type claims and features of the method type claims is considered to be disclosed with this application.

The aspects defined above and further aspects of the present invention are apparent from the examples of embodiment to be described hereinafter and are explained with reference to the examples of embodiment. The invention will be described in more detail hereinafter with reference to examples of embodiment but to which the invention is not limited.

### BRIEF DESCRIPTION OF THE DRAWINGS

FIG. 1 shows a medical apparatus comprising a medical device, which is used for optically investigating tissue material being surrounded laterally with respect to the medical device.

FIG. 2 shows a medical apparatus comprising a medical device, which is equipped with a plurality of different optical fiber outlets being arranged at a side wall of the medical device.

FIG. 3 shows a medical device comprising a hollow shaft, wherein a biopsy element is movably accommodated, and optical fiber outlets, which are directed towards the interior of the hollow shaft.

FIG. 4 shows perspective illustration of a medical device being equipped with reflector elements, which are arranged at a lateral surface of the medical device.

FIG. 5 shows a cross sectional view and a longitudinal sectional view of the medical device shown in FIG. 4.

### DETAILED DESCRIPTION

The illustration in the drawing is schematically. It is noted that in different figures, similar or identical elements are provided with the same reference signs or with reference signs, which are different from the corresponding reference signs only within the first digit.

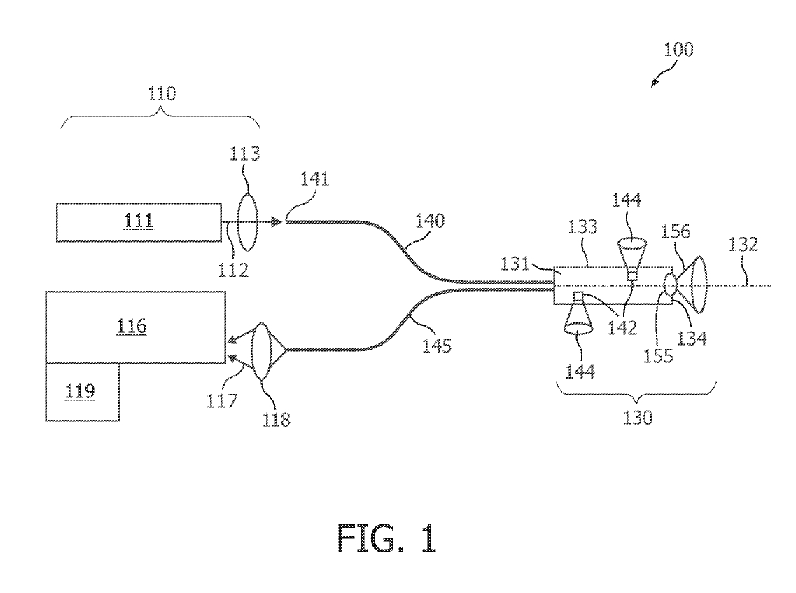

FIG. 1 shows a medical apparatus 100 according to a first embodiment of the present invention. The medical apparatus 100 comprises an optical instrument 110 and a medical device 130. According to the embodiment described here the medical device is an optical needle 130. The medical apparatus 100 is in particular suitable for optically investigating tissue material being surrounded laterally with respect to the medical device 130.

The optical instrument 110 comprises a light source 111, which is adapted to generate illumination light 112. According to the embodiment described here, the light source is a laser 111, which emits a monochromatic radiation beam 111. The radiation beam is directed via an optic 113 onto a first fiber end 141 of an optical fiber 140.

The optical instrument 110 further comprises a spectrometer device 116, which is optically coupled to an optical fiber 145 by means of an optic 118. The spectrometer device 116 is used for spectrally analyzing measurement light 117, which is provided by the medical device 130. The spectrometer device 116 is provided with a CCD camera 119 in order to detect measurement light 117, which is spectrally expanded by means of at least one refractive or diffractive optical element of the spectrometer device 116.

The medical device 130 comprises an elongated body 131 having a longitudinal axis 132. On a side wall 133 of the elongated body 131 there are provided second fiber ends 142, which are coupled to the optical fiber 140. The second fiber ends 142 are oriented in such a manner, that they provide each a lateral field of view 144, which might be used for illuminating tissue laterally surrounding the elongated body 131.

The medical device 130 further comprises a waveguide end, which is arranged at a front end 134 of the elongated body 131. The waveguide end 155 provides a front field of view 156, which is oriented substantially parallel to the longitudinal axis 132.

The two optical fibers 140 and 145 may be optically coupled to the lateral fiber outlets 142 and to the front waveguide outlet 155 in various combinations. Thereby, the outlets 142 and 155 may be coupled collectively or individually with the optical fiber 140 respectively the optical fiber 145. In this respect it is pointed out that the outlets, which are optically coupled to the optical fiber 145 respectively the spectrometer device 116 represent de facto an optical inlet, because measurement light, which has been scattered by the tissue, can enter these inlet such that this measurement light can be analyzed by means of the spectrometer device 116.

According to the embodiment shown in FIG. 1 both lateral fiber outlets 142 are assigned to the same optical fiber 140. However, it may also be possible to use one separate optical fiber for each of the two lateral fiber outlets 142 and/or for the front waveguide outlet 155. Of course, also less or more than two lateral fiber outlets 142 might be provided at the side wall 133 of the elongated body 131.

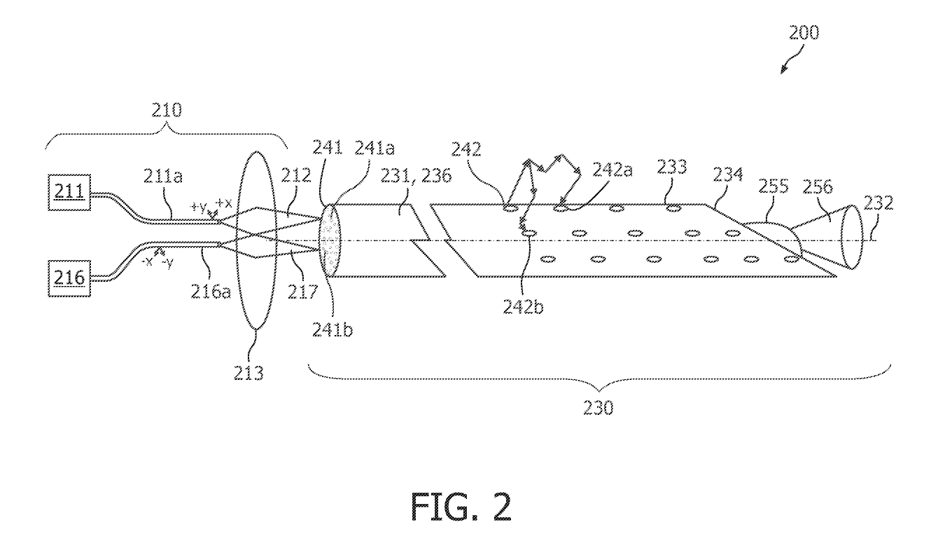

FIG. 2 shows a medical apparatus 200 according to a second embodiment of the present invention. The medical apparatus 200 comprises an optical instrument 210 and a medical device 230. According to the embodiment described here the medical device is a solid optical needle 130.

The optical instrument 210 comprises a light source 211, which is adapted to generate illumination light 212. The illumination light is guided by an optical fiber 211a, which may also be denominated an illumination fiber 211a. The optical instrument 210 further comprises a spectrometer device 216, which is adapted to receive a measurement light 217 by means of a measurement fiber 216a. An optic 213 is provided in order to optically couple the optical fiber 211a respectively the measurement fiber 216a with selected optical fibers being accommodated within the medical device 230.

The spectrometer device 216 may also be replaced with an optical detector 216 solely measuring the light intensity. The detector 216 may be equipped with a spectral filter in order to select a certain wavelength or a spectral range of the measurement light 217.

In order to selectively couple the illumination fiber 211a and/or the measurement fiber 216a with predetermined optical fibers of the medical device 230, there is provided a non depicted positioning system for adjusting ends of the illumination fiber 211a and/or the measurement fiber 216a in an x-y plane being oriented perpendicular to the longitudinal axis 232 of the optical needle 230.

The optical needle 230 comprises an elongated body 231 having a longitudinal axis 232. The elongated body 231 is a solid shaft 236, which accommodates a plurality of optical fibers. A front end 234 of the elongated body 231 is sharpened such that the medical device can be inserted into a patient's body without causing significant lesion to the patient.

On a side wall 233 of the elongated body 231 there are provided a plurality of second fiber ends 242, 242a, 242b. Each of the second fiber ends 242, 242a, 242b is optically connected by means of an optical fiber to a corresponding first fiber end 241, 241a, 241b. The second fiber ends 242, 242a, 242b are oriented in such a manner, that they each provide a lateral field of view, which might be used for illuminating tissue laterally surrounding the elongated body 231 and/or for receiving measurement light, which has been scattered by this tissue.

The medical device 230 further comprises a central waveguide, which extends to a waveguide end 255 at the sharpened distal end 234 of the elongated body 231. The waveguide end 255 provides a front field of view 256, which is oriented substantially parallel to the longitudinal axis 232.

As can be seen from FIG. 2, the optical needle 230 contains a collection of optical fibers without having a lumen. Each of the fiber entrance positions 241, 241a, 241b at the base of the needle is assigned to a lateral fiber outlet 242, 242a, 242b at the side wall 233 of the needle 230. In this way the needle 230 is equipped with a variety of different optical probe positions.

Light is coupled selectively into and out of the optical fibers at the base of the needle 230 by means of the optical instrument 210 described above. The light source 211, which is connected to the illumination fiber 211a, illuminates for instance the first fiber end 241. The light will cross the corresponding fiber and illuminate the tissue around the lateral outlet position 242. Light scattering from this position 242 can for instance reach the position 242a and 242b, which then represent a lateral fiber input. The detector 216 is connected to the measurement fiber 216a that collects the light coming from each first fiber end 241, 241a and 241b, respectively. The intensity of the measurement light 217 is a measure for the amount of absorption and scatter between the lateral outlet positions 242, 242a and 242b. From these signals the tissue characteristics around the needle can be extracted.

It is worth noting that the embodiment depicted in FIG. 2 allows a three-dimensional imaging of scattering and absorption properties of the tissue surrounding the needle 230.

Thereby, a longitudinal spatial resolution equal to that of the longitudinal fiber-to-fiber distance can be achieved.

It has to be mentioned that the described medical device 230 also allows for performing diffuse optical tomography (DOT) around the needle. This allows functional imaging in a relatively large volume around the needle. Thereby, one or more lateral fiber outlets 242, 242a, 242b are used for (sequential) illumination of the tissue. One or more other fibers outlets 242, 242a, 242b are used to collect the scattered light. Using an image reconstruction algorithm it is possible to obtain a 3D map of the optical tissue properties in a region around the needle 230. The main advantage of DOT is the high penetration depth compared to other optical methods. The penetration depth is about half of the distance between the source 242 and the detector 242a respectively 242b.

The most advantageous wavelength region for DOT is the near infrared (NIR) spectral regime. Here the penetration depth is at its maximum and the optical properties are strongly determined by important physiologic parameters like blood content and oxygen saturation. By combining DOT at different wavelengths it is possible to reliably translate optical parameters into physiological parameters.

Moreover, one can also perform an optical coherence scan for each fiber, which gives for each fiber a depth-scan along a line. Combining these lines, one can reconstruct a three-dimensional image of the tissue around the needle, again with a longitudinal resolution equal to that of the fiber-to-fiber distance.

In the following there will be briefly described one variation of this embodiment, wherein fluorescence imaging and/or spectroscopic measurements are implemented. Thereby, the light source 211 and the fiber 211a are used for exciting fluorescent molecules or atoms within the tissue. The corresponding fluorescence light being emitted by the molecules is collected and guided by means of the fiber 216a to the detector 216.

According to a further variation one can perform Raman spectroscopy. Thereby, the corresponding Raman spectroscopic data can be acquired separately for each fiber end position 242, 242a, 242b, etc.

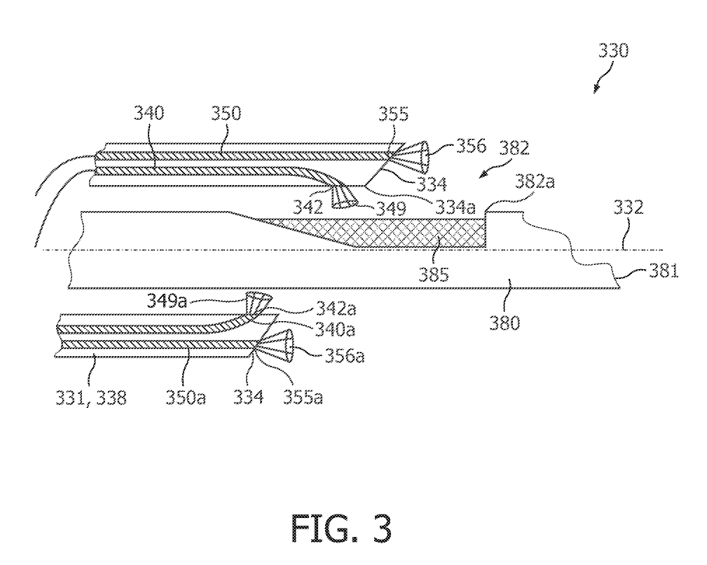

FIG. 3 shows a medical device 330, which comprises an elongated body 331. The elongated body 331 has the shape of a hollow shaft 338. A biopsy element 380 is movably accommodated within the hollow shaft 338 along a longitudinal axis 332 of the medical device 330. A front end 334 is sharpened in order to facilitate the insertion of the medical device 330 into a patient's body.

The biopsy element 380, which also comprises a sharpened distal end 381, comprises a recess 382 for collecting tissue specimen 385. The tissue specimen is also denoted a biopt 385. For collecting the biopt 385 the biopsy element 380 is moved towards the front end 334 such that the recess 382 protrudes from front end 334 of the hollow shaft 338. Upon moving the biopsy element 380 again inwardly a biopt, which has entered the recess 382, will be cut away from its neighboring tissue. The cut is carried out between the edge 382a and the edge 334a.

The shaft wall 338 contains optical fibers 340, 340a and optical waveguides 350, 350a. In the terminology used within these application the optical waveguides 350 and 350a are used for providing a front field of view 356 and 356a, respectively. By contrast thereto, the optical fibers 340 and 340a are used for providing a lateral field of view 349 and 349a, respectively. The lateral field of view 349 and the further lateral field of view 349a is originating from a second fiber end representing a lateral fiber outlet 342 and a further second fiber end representing a further lateral fiber outlet 342a, respectively.

As can be seen from FIG. 3, the lateral field of view 349 and 349a is directed inwardly such that the biopt 385, which has been removed from the patient's body, can be optically investigated immediately after the removal of the biopt 385. This means that the biopt 385 can be optically inspected before it is removed through the hollow shaft 338 to the outside world. In this way one can inspect whether the biopt 385 is of good quality and whether it contains sufficient tissue prior to removal. If this is not the case, a new biopsy can be carried out immediately because the lumen of the recess 382 respectively the hollow shaft 338 consists of enough space to allow more than one biopsy to be accomplished.

As can be further seen from FIG. 3, the front field of view 356 and the further front field of view 356a originating from a second waveguide end 355 and a further second 355a, respectively, are directed substantially parallel to the longitudinal axis 332. This provides the advantage that tissue being located in front of the sharpened distal end 334 can be illuminated. At least a part of the resulting scattered and emitted light is collected by other optical fibers and guided to a spectrograph, where for instance a Raman spectrum is recorded. Upon inspection of the spectrum it can be decided either to carry out a biopsy or to further move the shaft 338 through the patient's tissue in order to reach another position at which anomalies in the spectrum are found. Such anomalies can indicate for instance a malign tissue, which, in order to provide a reliable positive or negative cancer diagnosis, is very important to be investigated by a pathologist.

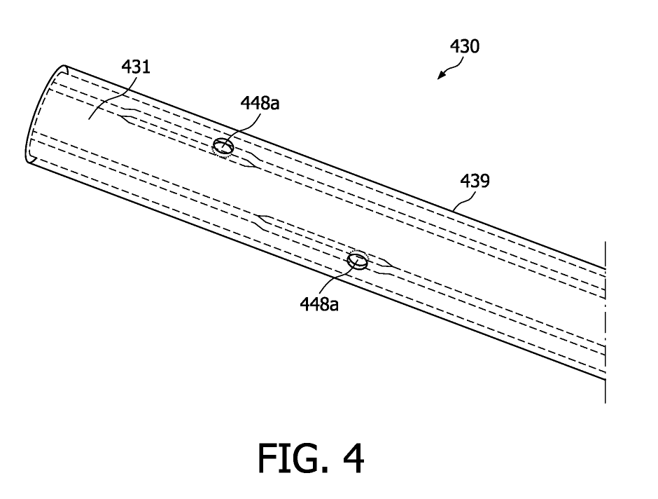

FIG. 4 shows perspective illustration of a medical device 430 being equipped with reflector elements 448a. The reflector elements 448a, which are arranged at a side wall of the elongated body 431, are each coupled to an optical fiber being accommodated within the elongated body 431. Each reflector element 448a is used either for reflecting illumination light, which is emitted from a second fiber end of the optical fiber, or for reflecting measurement light, which is scattered or emitted from the tissue laterally surrounding a housing 439 of the elongated body 431. The housing 439 is used in order to mechanically protect the medical device 430. According to the embodiment depicted here, the housing 439 is made from a transparent material. However, it has to be mentioned that it is also possible to manufacture the medical device 430 with an optically opaque housing.

The reflector elements 448a provide the advantage that the corresponding field of view of each optical fiber being equipped with a reflector element 448a can be oriented substantially perpendicular to the longitudinal axis of the elongated body 431 without bending the corresponding optical fibers.

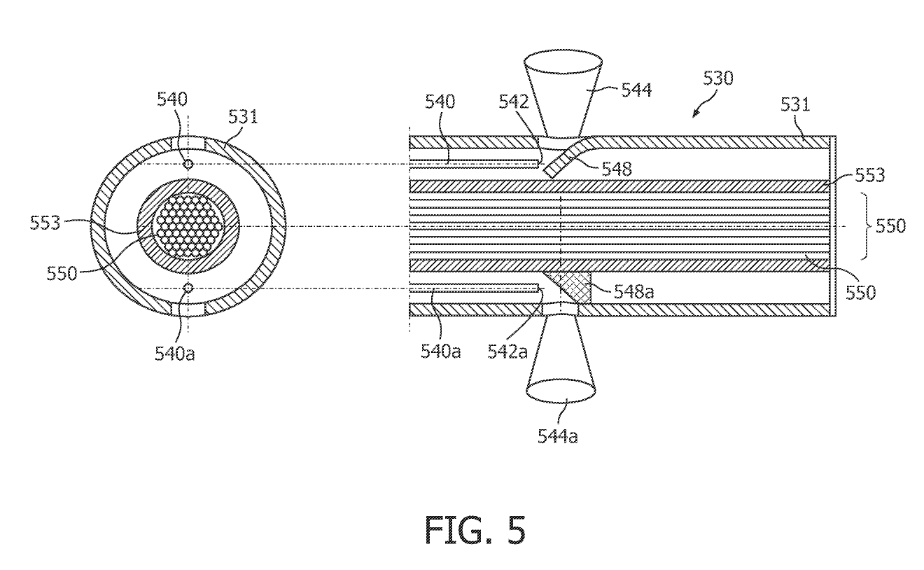

FIG. 5 shows a cross sectional view (left side) and a longitudinal sectional view (right side) of the medical device shown in FIG. 4, which is now denoted with reference numeral 530. The medical device comprises an elongated body 531, which accommodates an optical fiber 540 and a further optical fiber 540a. The optical fiber 540 comprises a second fiber end 542. The further optical fiber 540a comprises a further second fiber end 542a. A lateral field of view 544 is assigned to second fiber end 542. A further lateral field of view 544a is assigned to the further second fiber end 542a.

In order to laterally direct the field of view 544, 544a radially outward without having the need to bend the optical fibers 540, 540a, reflector elements 548, 548a are employed. In the right view of the medical device 530 shown in FIG. 5 two possibilities for realizing the reflector elements 548, 548a are illustrated.

The reflector elements may be for instance realized by means of a mirror element 548. The mirror element 548 can be formed integrally with the shaft wall of the elongated body 531. Alternatively, the reflector elements may be realized by prisms 548a, which are attached to close to an opening of the shaft wall of the elongated body 531.

The elongated body 531 further accommodates an inner housing 553, which itself accommodates a waveguide 550. As can be seen in particular from the left view shown in FIG. 5, the accommodated waveguide 550 comprises a bundle of optical fibers elements. As has already been explained above in detail, the waveguide 550 is used in order to provide for a not depicted front field of view of the medical device 530.

It should be noted that the term “comprising” does not exclude other elements or steps and the “a” or “an” does not exclude a plurality. Also elements described in association with different embodiments may be combined. It should also be noted that reference signs in the claims should not be construed as limiting the scope of the claims.

In order to recapitulate the above described embodiments of the present invention one can state:

This application describes a medical device 230 for obtaining optical tissue properties of a target material. The medical device 230 comprises an elongated body 231 having a longitudinal axis 232 and an optical fiber being integrated within the elongated body 231. The optical fiber has a second fiber end 242, 242a, 242b, which is arranged at a side wall 233 of the elongated body 231 and which provides a lateral field of view with respect to the longitudinal axis 232.

According to an embodiment many optical fibers are integrated each having an optical outlet 242, 242a, 242b around the elongated body 231. Using the outlets 242, 242a, 242b to do diffuse optical tomography (DOT) and also use optical fibers to do optical inspection like optical coherence tomography, Raman spectroscopy, light scattering spectroscopy etc., one can get information on the presence of tumors in a volume around the medical device 230 via DOT (few cm) and a tissue characterization in the vicinity of the medical device 230 (few 100 microns). This is interesting in particular for prostate cancer. DOT finds suspicious areas in prostate, by guiding the medical device 230 closer to these suspicious areas, whereby based on the optical techniques precise diagnosis can be made. Thereby, a DOT guided optical biopsy may be carried out, wherein no real tissue is removed.

According to another embodiment an optical detection system is integrated into a real biopsy needle 330 allowing inspection and taking real biopsy simultaneously.

### LIST OF REFERENCE SIGNS

100 medical apparatus

110 optical instrument

111 light source/laser

112 illumination light/radiation beam

113 optic

116 spectrometer device

117 measurement light

118 optic

119 CCD camera

130 medical device/optical needle

131 elongated body

132 longitudinal axis

133 side wall

134 front end

140 optical fiber

141 first fiber end

142 second fiber end/lateral fiber outlet

144 lateral field of view

145 optical fiber

155 waveguide end/front waveguide outlet

156 front field of view

200 medical apparatus

210 optical instrument

211 light source

211a illumination fiber

212 illumination light

213 optic

216 spectrometer device/detector

216a measurement fiber

217 measurement light

230 medical device/optical needle

231 elongated body

232 longitudinal axis

233 side wall

234 front end/sharpened distal end

236 solid shaft

241 first fiber end, first fiber entrance position

241a/b further first fiber end, further fiber entrance positions

242 second fiber end/lateral fiber outlet/lateral outlet position

242a/b further second fiber end/further lateral fiber outlet/further lateral outlet position

255 waveguide end

256 front field of view

+/−x x-direction

+/−y y-direction

330 medical device/optical needle

331 elongated body

332 longitudinal axis

334 front end/sharpened distal end

334a edge

338 hollow shaft/shaft wall

340 optical fiber

340a further optical fiber

342 second fiber end/lateral fiber outlet

342a further second fiber end/further lateral fiber outlet

349 interior lateral field of view

350 optical waveguide

350a further optical waveguide

355 second waveguide end

355a further second waveguide end

356 front field of view

356a further front field of view

380 biopsy element

381 sharpened distal end

382 recess

382a edge

385 specimen/biopt

430 medical device/optical needle

431 elongated body

439 housing

448a reflector element/prism

530 medical device/optical needle

531 elongated body

540 optical fiber

540a further optical fiber

542 second fiber end

542a further second fiber end

544 lateral field of view

544a further lateral field of view

548 reflector element/mirror element

548a reflector element/prism

550 waveguide comprising bundle of optical fiber elements

553 inner housing

1. A medical device, in particular a needle (230, 330), for obtaining optical tissue properties of a human or animal body, the medical device (230, 330) comprising
an elongated body (231, 331) having a longitudinal axis (232, 332), wherein the elongated body (231, 331) is designed to be insertable into tissue of a human or an animal body, and
an optical fiber (340) being integrated within the elongated body (231, 331), the optical fiber (340) having a first fiber end (241) and a second fiber end (242, 342), wherein the first fiber end (241) is adapted to be coupled to an optical instrument (210), the second fiber end (242, 342) is arranged at a side wall (233, 338) of the elongated body (231, 331) and
the second fiber (242, 342) end provides a lateral field of view (144, 349), which is directed in a lateral direction with respect to the longitudinal axis (232, 332).

2. The medical device according to claim 1, further comprising
a reflector element (448a, 548, 548a), which is arranged at the side wall (233, 338) of the elongated body (231, 331,531) and which is optically coupled to the second fiber end (242, 342) of the optical fiber (340).

3. The medical device according to claim 1, wherein
the elongated body (231, 331) comprises a sharpened distal end (234, 334).

4. The medical device according to claim 1, further comprising
an optical waveguide (350) being integrated within the elongated body (231, 331), the optical waveguide (350) having a first waveguide end and a second waveguide end (255, 355), wherein
the first waveguide end is adapted to be coupled to an optical instrument (210), the second waveguide end (255, 355) is arranged at a front end (234, 334) of the elongated body (231, 331) and
the second waveguide end (255, 355) provides a front field of view (256, 356), which is directed in a longitudinal direction with respect to the longitudinal axis (232, 332).

5. The medical device according to claim 1, further comprising
at least one further optical fiber (340a) being integrated within the elongated body (331), the further optical fiber (340a) having a further first fiber end (241a) and a further second fiber end (242a, 342a), wherein
the further first fiber end (241a) is adapted to be coupled to an optical instrument (210), the further second fiber end (242a, 342a) is arranged at a side wall (233, 338) of the elongated body (231, 331) and
the further second fiber end (242a, 342a) provides a further lateral field of view (349a), which is directed in a lateral direction with respect to the longitudinal axis (232, 332).

6. The medical device according to claim 1, wherein
the elongated body (231) is a solid shaft (236).

7. The medical device according to claim 1, wherein
the elongated body (331) is a hollow shaft (338).

8. The medical device according to claim 7, further comprising
a biopsy element (381) being movably accommodated within the hollow shaft (338).

9. The medical device according to claim 7, wherein
the second fiber end (342a) provides an interior lateral field of view (349a), which is directed from the shaft wall (338) towards the central longitudinal axis of the hollow shaft (338).

10. A medical apparatus for obtaining optical tissue properties of a human or animal body, the medical apparatus (100, 200) comprising
a medical device (130, 230, 330) according to claim 1 and
an optical instrument (110, 210), which is optically coupled to the optical fiber (140, 340) of the medical device (130, 230, 330).

11. The medical apparatus according to claim 10, wherein
the optical instrument (110, 210) comprises
a light source (111, 211), which is adapted to generate illumination light (112, 212) for being injected into the optical fiber (140, 340), and
an optical detector (116, 216), which is adapted to receive measurement light (117, 217) being transmitted by the optical fiber (140, 340).

12. The medical apparatus according to claim 10, wherein
the optical instrument (110, 210) is adapted to perform diffused optical tomography and/or
the optical instrument (110, 210) is adapted to perform optical coherence tomography.

13. The medical apparatus according to claim 10, wherein
the optical instrument (110, 210) is adapted to perform at least one of the following optical procedures:
Raman spectroscopy, fluorescence spectroscopy, auto fluorescence spectroscopy, two-photon spectroscopy, and differential path length spectroscopy.

14. A method for obtaining optical tissue properties of a human or animal body, the method comprising
illuminating the tissue with illumination light (112, 212), which has been emitted from a light source (111, 211) and which has been transmitted by means of a medical device (130, 230, 330) according to claim 1, and
detecting measurement light (117, 217), which has interacted with the tissue and which has been transmitted by means of the medical device (130, 230, 330).

15. The method according to claim 14, further comprising
applying a photosensitive agent.
